(19) United States
(12) Patent Application Publication
(10) Pub. No.: US 2023/0026775 A1
(43) Pub. Date: Jan. 26, 2023

(54) DETECTION AND SEQUENCING OF FRAGMENTED DNA

(71) Applicant: MEMORIAL SLOAN KETTERING CANCER CENTER, New York, NY (US)

(72) Inventors: Daniel Smith Higginson, Tenafly, NJ (US); Rama Rao Damerla, Manipal (IN)

(21) Appl. No.: 17/777,719

(22) PCT Filed: Nov. 18, 2020
(86) PCT No.: PCT/US20/61038

§ 371 Date: May 18, 2022

Related U.S. Application Data
(60) Provisional application No. 62/936,832, filed on Nov. 18, 2019.

(51) Int. Cl. C12N 15/10; C12Q 1/70

(52) U.S. Cl. CPC C12N 15/1068; C12Q 1/708

(57) ABSTRACT

The present invention provides modified single primer extension-based methods for generating an amplified library of fragments of a target gene or genome of interest from a sample of fragmented DNA, wherein the library is suitable for use in detecting, quantifying and/or sequencing the target gene or genome of interest. The present invention also provides compositions for use in such methods. In some embodiments the present invention provides methods and compositions specifically for detecting, quantifying and/or sequencing circulating tumor derived HPV DNA.

### CROSS-REFERENCE TO RELATED APPLICATIONS

This application claims the benefit of priority of U.S. Provisional Patent Application No. 62/936,832 filed on Nov. 18, 2019, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 18, 2020, is named MSKCC_037_WO1_SL.txt and is 17,659 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

Circulating cell-free DNA (cfDNA) is fragmented DNA present in the vascular circulation (i.e. in the plasma), as well as other bodily fluids. In healthy individuals the levels of cfDNA are generally low. However, during pregnancy and illness levels of cfDNA increase. For example, during pregnancy cfDNA derived from the fetus (cell-free fetal DNA or cffDNA) is often present circulating in maternal plasma and has been utilized in pre-natal screening. In cancer patients, circulating tumor DNA (ctDNA) can be detected in the plasma.

Circulating cell-free DNA (cfDNA), including circulating tumor-derived DNA (ctDNA), is an emerging biomarker category. In the case of ctDNA, detection of virally-derived ctDNA (i.e. from virally-driven tumors) is particularly useful as a biomarker, because being virally derived rather than host derived, the signal to noise ratio is improved. Similarly, because there are often multiple copies of viral DNA within one cancer cell, the amount of virally-derived ctDNA in the blood is often much higher than that of other ctDNAs.

ctDNA detection of tumor derived Epstein-Barr Virus (EBV) DNA has been successful for early detection of nasopharyngeal cancer (NPC)⁴ and persistent EBV ctDNA levels are a negative predictive factor for recurrence following chemoradiation.⁵⁻⁸

Cancers driven by human papillomaviruses (HPV) include squamous cell carcinomas of the oropharynx, cervix, vulva, vagina, anal canal and penis. While Papanicolaou (Pap) smears are widely used for early detection of HPV-associated lesions in the cervix, effective screening approaches for other HPV-associated cancers are lacking. For example, HPV-associated oropharyngeal cancers (HPV+OPSCCs) represent a large cohort of HPV-associated cancers for which there is currently no effective screening paradigm. While some studies have demonstrated detection of HPV plasma ctDNA, to date such studies have generally shown only modest sensitivity.

There is a need in the art for new and improved methods for detecting, measuring, and sequencing cfDNA and ctDNA, including, but not limited to, HPV ctDNA. The present invention addresses these needs.

### SUMMARY OF THE INVENTION

The present invention provides new and improved methods for capturing, detecting, measuring, amplifying and/or sequencing fragmented DNA such as cfDNA and ctDNA, including, but not limited to, HPV ctDNA.

In some aspects the present invention provides novel library preparation methods useful for capturing, detecting, quantifying, amplifying and/or sequencing fragmented DNA. These methods utilize a PCR-based approach and generate libraries that are amenable to analysis by sequencing using a next generation sequencing approach. The utility of these methods is exemplified herein using ctDNA from HPV-associated tumors. However, the technology has broader applicability—being useful for capturing, amplifying, detecting, quantifying and/or sequencing cfDNA from several tumor types and other disease types, cfDNA (including cffDNA), and other types of fragmented DNA.

Conventional PCR-based approaches for the amplification of a DNA target sequence require the use of two primers—a forward primer and a reverse primer—each of which has a sequence that is complementary to, and can bind to, a primer binding site in the DNA target sequence. The primer binding sites are separated from one another—i.e. they are a certain distance apart on the DNA target sequence. The distance between the primer binding sites determines the size of the amplicon generated when these two primers are used in a PCR reaction. As such, conventional PCR-based methods have limited utility for the detection of fragmented DNA (such as cfDNA and ctDNA) because a given fragmented DNA molecule may not contain the binding sites for both the forward and reverse primers. To address this issue, single primer extension or “SPEX” techniques were developed—originally for the detection of fragmented ancient DNA (1). The methods of the present invention involve certain improvements to, and modifications of, prior SPEX methods. These improved methods—which are referred to herein as “Single Primer Extension CTailing and Reverse Extension (SPECTRE-seq)” methods—are expected to vastly improve the accuracy and sensitivity of PCR-based and next-generation sequencing (NGS)-based methods for detection of fragmented DNA. As described herein, SPECTRE-seq methods can “capture” the entire HPV genome in a ctDNA sample so that it can be accurately detected, quantified and sequenced. SPECTRE-seq can also be applied to investigate any genomic target from various sources of fragmented DNA—whether cfDNA, ctDNA, or fragmented DNA from any other source.

These methods, as well as various compositions useful in performing such methods, are further described in subsequent sections of this patent disclosure including the Detailed Description, Examples, Claims and Drawings sections—each of which sections is intended to be read in conjunction with, and in the context of, all other sections of the present patent disclosure. Furthermore, one of skill in the art will recognize that the various embodiments of the present invention described herein can be combined in various ways, and that such combinations are within the scope of the present invention.

### BRIEF DESCRIPTION OF THE DRAWINGS

FIG. 1 Schematic diagram of an exemplary SPECTRE-seq method of the present invention. Rows A-F in the diagram represent individual steps in the method.

FIG. 2. Schematic diagram of an exemplary SPECTRE-seq method of the present invention. Rows A-D in the diagram represent individual steps in the method.

FIG. 3A-C. Optimization of SPECTRE-seq with ssDNA template. FIG. 3A is a schematic diagram of an exemplary SPECTRE-seq method of the present invention. FIG. 3B is a gel image. The numbers on each lane correspond to the molecules on the gel image as represented in A. SPECTRE extension primer (1), single stranded (ssDNA) template used for extension of SPECTRE primer (2), extension products (3), C-tailing of extension product (4), second or reverse extension of c-tailed products (5). FIG. 3C provides a table representing the results of Sanger sequencing of 10 colonies from TOPO cloned SPECTRE products. The molecule represented above the table is a schematic of expected product from SPECTRE experiment. 8 out of 10 colonies showed expected products with extension, c-tailing and incorporation of sequencing adapters indicating proof of principle of this technique.

### DETAILED DESCRIPTION OF THE INVENTION

### Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges provided herein are inclusive of the numbers defining the range.

Where a numeric term is preceded by “about” or “approximately,” the term includes the stated number and values ±20% of the stated number.

Numbers in parentheses or superscript following text in this patent disclosure refer to the numbered references provided in the “Reference List” section at the end of this patent disclosure.

Wherever embodiments are described with the language “comprising,” otherwise analogous embodiments described in terms of “consisting of” and/or “consisting essentially of” are included.

Other abbreviations and definitions may be provided elsewhere in this patent specification, or may be well known in the art.

### Methods

The present invention provides modified single primer extension-based methods for generating an amplified library of fragments of a target gene or genome of interest from a sample of fragmented DNA. Such libraries are useful for detecting, quantifying and/or sequencing such a target gene or genome of interest. The present invention also provides various compositions for use in such methods. In some embodiments the methods and compositions provided herein are designed specifically for, and are useful for, detecting, quantifying and/or sequencing circulating tumor DNA, such as circulating tumor derived HPV DNA (i.e. from HPV-associated or HPV-driven tumors).

Accordingly, in one embodiment the present invention provides a method of generating a library of fragments of a target gene or genome of interest from a sample of fragmented DNA, wherein the library is suitable for use in detecting, quantifying and/or sequencing the target gene or genome of interest, the method comprising: (a) contacting a sample of fragmented DNA with a pool of target-specific forward primers complementary to multiple different primer binding sites located within, and spanning the length of, a target gene or genome of interest, wherein each target-specific forward primer comprises: (i) a sequence that is complementary to a primer binding site within the target gene or genome of interest and (ii) a first next generation sequencing (NGS) based adapter located 5′ to the sequence that is complementary to the primer binding site, (b) performing a single primer extension reaction to generate first-generation copies of the target gene of genome of interest, (c) performing a nucleotide tailing reaction or adding a common sequence to the 3′ end of the first-generation copies of the target gene or genome of interest, thereby generating 3′ tagged first generation copies of the fragmented target gene or genome of interest, (d) performing a first PCR reaction using: a common reverse primer comprising: (i) a sequence that is complementary to the common sequence and (ii) a second next generation sequencing (NGS) based adapter located 5′ to the sequence that is complementary to the common sequence, and (e) performing a second PCR reaction using: (i) a forward primer complementary to the NGS-based adapter present in the target-specific forward primer, and (ii) a reverse primer complementary to the NGS-based adapter present the common reverse primer, thereby generating a library of fragments of the target gene or genome of interest from a sample of fragmented DNA, wherein the library is suitable for use in detecting, quantifying and/or sequencing the target gene or genome of interest.

In some embodiments the present invention provides a variation of the above method, in which the single primer extension reaction of step (b) is performed in the presence of biotinylated nucleotides—such that the first-generation copies of the target gene of genome of interest generated by the single primer extension reaction are biotinylated. This enables a biotin-based selection/purification step to be performed to select for only the single primer extension products before proceeding with subsequent steps of the method. Any suitable biotin-based selection method can be used. For example, the products of the single primer extension reaction can be contacted with a streptavidin-coated solid support (e.g. beads or a column) to which the biotinylated products will bind and can be eluted—using methods well known in the art.

The libraries of fragments of the target gene or genome of interest generated using the methods of the present invention can be analyzed in various ways to facilitate the detection, quantification, and/or sequencing of the target gene or genome of interest. For example, in some embodiments the libraries of fragments of the target gene or genome of interest generated using the methods of the present invention can be analyzed by performing quantitative PCR (qPCR). In some embodiments the libraries of fragments of the target gene or genome of interest generated using the methods of the present invention can be analyzed by performing sequencing, such as next generation sequencing.

The sample of fragmented DNA used in the methods of the present invention can be any suitable source of fragmented DNA. In one embodiment the fragmented DNA is, or comprises, circulating cell free DNA (cfDNA). In another embodiment the fragmented DNA is, or comprises, circulating tumor DNA (ctDNA).

In some embodiments the pool of target-specific forward primers used in the methods of the present invention comprises primers complementary to from tens to up to thousands different primer binding sites within the target gene or genome of interest (e.g. approximately 10, or 20, or 30, or 40, or 50, or 60, or 70, or 75, or 80, or 90, or 100, or 200, or 300, or 400, or 500, or 600, or 700, or 800, or 900, or 1000, or 1250, or 1500, or 1750, or 2000, or 3000, or 4000, or 5000 different primer binding sites within the target gene or genome of interest). For example, in one embodiment the pool of target-specific forward primers comprises primers complementary to approximately 10 different primer binding sites within the target gene or genome of interest. In one embodiment the pool of target-specific forward primers comprises primers complementary to approximately 20 different primer binding sites within the target gene or genome of interest. In one embodiment the pool of target-specific forward primers comprises primers complementary to approximately 30 different primer binding sites within the target gene or genome of interest. In one embodiment the pool of target-specific forward primers comprises primers complementary to approximately 40 different primer binding sites within the target gene or genome of interest. In one embodiment the pool of target-specific forward primers comprises primers complementary to approximately 50 different primer binding sites within the target gene or genome of interest. In one embodiment the pool of target-specific forward primers comprises primers complementary to approximately 60 different primer binding sites within the target gene or genome of interest. In one embodiment the pool of target-specific forward primers comprises primers complementary to approximately 70 different primer binding sites within the target gene or genome of interest. In one embodiment the pool of target-specific forward primers comprises primers complementary to approximately 75 different primer binding sites within the target gene or genome of interest. In one embodiment the pool of target-specific forward primers comprises primers complementary to approximately 80 different primer binding sites within the target gene or genome of interest. In one embodiment the pool of target-specific forward primers comprises primers complementary to approximately 90 different primer binding sites within the target gene or genome of interest. In one embodiment the pool of target-specific forward primers comprises primers complementary to approximately 100 different primer binding sites within the target gene or genome of interest. In one embodiment the pool of target-specific forward primers comprises primers complementary to approximately 200 different primer binding sites within the target gene or genome of interest. In one embodiment the pool of target-specific forward primers comprises primers complementary to approximately 300 different primer binding sites within the target gene or genome of interest. In one embodiment the pool of target-specific forward primers comprises primers complementary to approximately 400 different primer binding sites within the target gene or genome of interest. In one embodiment the pool of target-specific forward primers comprises primers complementary to approximately 500 different primer binding sites within the target gene or genome of interest. In one embodiment the pool of target-specific forward primers comprises primers complementary to approximately 600 different primer binding sites within the target gene or genome of interest. In one embodiment the pool of target-specific forward primers comprises primers complementary to approximately 700 different primer binding sites within the target gene or genome of interest. In one embodiment the pool of target-specific forward primers comprises primers complementary to approximately 800 different primer binding sites within the target gene or genome of interest. In one embodiment the pool of target-specific forward primers comprises primers complementary to approximately 900 different primer binding sites within the target gene or genome of interest. In one embodiment the pool of target-specific forward primers comprises primers complementary to approximately 1,000 different primer binding sites within the target gene or genome of interest. In another embodiment the pool of target-specific forward primers comprises primers complementary to approximately 2,000 different primer binding sites within the target gene or genome of interest. In another embodiment the pool of target-specific forward primers comprises primers complementary to approximately 3,000 different primer binding sites within the target gene or genome of interest. In another embodiment the pool of target-specific forward primers comprises primers complementary to approximately 4,000 different primer binding sites within the target gene or genome of interest. In another embodiment the pool of target-specific forward primers comprises primers complementary to approximately 5,000 different primer binding sites within the target gene or genome of interest. In some embodiments the pool of target-specific forward primers comprises primers complementary to from about 50 to about 100 different primer binding sites within the target gene or genome of interest. In some embodiments the pool of target-specific forward primers comprises primers complementary to from about 60 to about 90 different primer binding sites within the target gene or genome of interest. In some embodiments the pool of target-specific forward primers comprises primers complementary to from about 70 to about 80 different primer binding sites within the target gene or genome of interest.

In some embodiments the different primer binding sites within the target gene or genome of interest to which the pool of target-specific forward primers is complementary are spaced approximately 20 to 200 nucleotides apart (e.g. approximately 20, or 30, or 40, or 50, or 60, or 70, or 80, or 90, or 110, or 120, or 140, or 160, or 180, or 200 nucleotides apart).

The number of cycles of the single primer extension reaction that is performed can be selected as desired. For example, in some embodiments from 1 to about 99 cycles of the single primer extension reaction are performed. In some embodiments about 10 cycles of the single primer extension reaction are performed. In some embodiments about 20 cycles of the single primer extension reaction are performed. In some embodiments about 30 cycles of the single primer extension reaction are performed. In some embodiments about 40 cycles of the single primer extension reaction are performed. In some embodiments about 50 cycles of the single primer extension reaction are performed. In some embodiments about 60 cycles of the single primer extension reaction are performed. In some embodiments about 70 cycles of the single primer extension reaction are performed. In some embodiments about 80 cycles of the single primer extension reaction are performed. In some embodiments about 90 cycles of the single primer extension reaction are performed. In some embodiments about 100 cycles of the single primer extension reaction are performed.

The number of cycles of the first PCR reaction that is performed can be selected as desired. For example, in some embodiments from 1 to about 99 cycles of the first PCR reaction are performed. In some embodiments about 10 cycles of the first PCR reaction are performed. In some embodiments about 20 cycles of the first PCR reaction are performed. In some embodiments about 30 cycles of the first PCR reaction are performed. In some embodiments about 40 cycles of the first PCR reaction are performed. In some embodiments about 50 cycles of the first PCR reaction are performed. In some embodiments about 60 cycles of the first PCR reaction are performed. In some embodiments about 70 cycles of the first PCR reaction are performed. In some embodiments about 80 cycles of the first PCR reaction are performed. In some embodiments about 90 cycles of the first PCR reaction are performed. In some embodiments about 100 cycles of the first PCR reaction are performed.

The number of cycles of the second PCR reaction that is performed can be selected as desired. For example, in some embodiments from 1 to about 99 cycles of the second PCR reaction are performed. In some embodiments about 10 cycles of the second PCR reaction are performed. In some embodiments about 20 cycles of the second PCR reaction are performed. In some embodiments about 30 cycles of the second PCR reaction are performed. In some embodiments about 40 cycles of the second PCR reaction are performed. In some embodiments about 50 cycles of the second PCR reaction are performed. In some embodiments about 60 cycles of the second PCR reaction are performed. In some embodiments about 70 cycles of the second PCR reaction are performed. In some embodiments about 80 cycles of the second PCR reaction are performed. In some embodiments about 90 cycles of the second PCR reaction are performed. In some embodiments about 100 cycles of the second PCR reaction are performed.

Any suitable next generation sequencing (NGS) based adapters in can be used in the methods of the invention. In some embodiments an Illumina NGS based adapter is used.

In some embodiments the “common sequence” used in the methods of the present invention is any suitable polynucleotide sequence. In some embodiments the “common sequence” is a polyC sequence. In some embodiments the “common sequence” is a polyG sequence. In some embodiments the “common sequence” is a polyA sequence. In some embodiments the “common sequence” is a polyT sequence.

In those embodiments of the present invention that utilize biotinylated nucleotides any suitable biotinylated nucleotides can be used. In some embodiments biotin-dCTP nucleotides are used. In some embodiments biotin-dGTP nucleotides are used. In some embodiments biotin-dATP nucleotides are used. In some embodiments any combination of biotin-dCTP, biotin-dGTP, biotin-dATP and/or biotin dTTP nucleotides are used.

In some embodiments the sample of fragmented used in the methods of the present invention is, or comprises, HPV circulating tumor DNA (ctDNA) and the target gene or genome of interest is an HPV gene or genome. In some such embodiments the HPV ctDNA is from an HPV-associated squamous cell carcinoma of the head and neck, oropharynx, cervix, vulva, vagina, anal canal or penis. In some such embodiments the HPV ctDNA is from an HPV-associated squamous cell carcinoma of the head and neck. In some such embodiments the HPV ctDNA is from an HPV-associated squamous cell carcinoma of the oropharynx. In some such embodiments the HPV ctDNA is from an HPV-associated squamous cell carcinoma of the cervix. In some such embodiments the HPV ctDNA is from an HPV-associated squamous cell carcinoma of the vulva. In some such embodiments the HPV ctDNA is from an HPV-associated squamous cell carcinoma of the vagina. In some such embodiments the HPV ctDNA is from an HPV-associated squamous cell carcinoma of the anal canal. In some such embodiments the HPV ctDNA is from an HPV-associated squamous cell carcinoma of the penis. Similarly, in some such embodiments the HPV ctDNA is from HPV type 16, 18, 31, 33, 35, 39, 45, 51, 52, 56, 58 or 59. For example, in some embodiments the HPV ctDNA is from HPV type 16. In some embodiments the HPV ctDNA is from HPV type 18. In some embodiments the HPV ctDNA is from HPV type 31. In some embodiments the HPV ctDNA is from HPV type 33. In some embodiments the HPV ctDNA is from HPV type 35. In some embodiments the HPV ctDNA is from HPV type 39. In some embodiments the HPV ctDNA is from HPV type 45. In some embodiments the HPV ctDNA is from HPV type 51. In some embodiments the HPV ctDNA is from HPV type 52. In some embodiments the HPV ctDNA is from HPV type 56. In some embodiments the HPV ctDNA is from HPV type 58. In some embodiments the HPV ctDNA is from HPV type 59.

In those embodiments of the present invention where the target gene or genome of interest is an HPV gene or genome, the methods provided herein utilize a pool of target-specific forward primers that are HPV-specific—i.e. a pool of HPV-specific forward primers. Examples of suitable HPV-specific forward primers are provided in Table 1, below—which provides the sequences of 77 different HPV-specific forward primers. For example, in some such embodiments the pool of target-specific forward primers comprises one or more of SEQ ID NO. 1 through SEQ ID NO. 77. In some such embodiments the pool of target-specific forward primers comprises at least 10 primers from among SEQ ID NO. 1 through SEQ ID NO. 77. In some such embodiments the pool of target-specific forward primers comprises at least 20 primers from among SEQ ID NO. 1 through SEQ ID NO. 77. In some such embodiments the pool of target-specific forward primers comprises at least 30 primers from among SEQ ID NO. 1 through SEQ ID NO. 77. In some such embodiments the pool of target-specific forward primers comprises at least 40 primers from among SEQ ID NO. 1 through SEQ ID NO. 77. In some such embodiments the pool of target-specific forward primers comprises at least 50 primers from among SEQ ID NO. 1 through SEQ ID NO. 77. In some such embodiments the pool of target-specific forward primers comprises at least 60 primers from among SEQ ID NO. 1 through SEQ ID NO. 77. In some such embodiments the pool of target-specific forward primers comprises at least 70 primers from among SEQ ID NO. 1 through SEQ ID NO. 77. In some such embodiments the pool of target-specific forward primers comprises each of SEQ ID NO. 1 through SEQ ID NO. 77. In some embodiments the pool of target-specific forward primers comprises primers that bind to a target sequence in the HPV genome that: (i) is within the E6 and/or E7 region of the HPV genome, and/or (ii) is 100% conserved between European and non-European HPV isolates.

### Compositions

The present invention also provides various compositions useful in performing the methods described herein. For example, the present invention provides compositions comprising a pool of target-specific forward primers suitable for use in a single primer extension reaction, wherein the pool comprises primers complementary to multiple different primer binding sites located within, and spanning the length of, a target gene or genome of interest, wherein each target-specific forward primer comprises: (i) a sequence that is complementary to a primer binding site within the target gene or genome of interest and (ii) a first next generation sequencing (NGS) based adapter located 5′ to the sequence that is complementary to the primer binding site.

In some such embodiments such compositions comprise a pool of target-specific forward primers complementary to a primer binding sites in circulating cell free DNA (cfDNA). In some such embodiments such compositions comprise a pool of target-specific forward primers complementary to a primer binding sites in circulating tumor DNA (ctDNA).

In some embodiments such compositions comprise a pool of target-specific forward primers that are complementary to from tens to up to thousands different primer binding sites within the target gene or genome of interest (e.g. approximately 10, or 20, or 30, or 40, or 50, or 60, or 70, or 75, or 80, or 90, or 100, or 200, or 300, or 400, or 500, or 600, or 700, or 800, or 900, or 1000, or 1250, or 1500, or 1750, or 2000, or 3000, or 4000, or 5000 different primer binding sites within the target gene or genome of interest). For example, in one embodiment the pool of target-specific forward primers comprises primers complementary to approximately 10 different primer binding sites within the target gene or genome of interest. In one embodiment the pool of target-specific forward primers comprises primers complementary to approximately 20 different primer binding sites within the target gene or genome of interest. In one embodiment the pool of target-specific forward primers comprises primers complementary to approximately 30 different primer binding sites within the target gene or genome of interest. In one embodiment the pool of target-specific forward primers comprises primers complementary to approximately 40 different primer binding sites within the target gene or genome of interest. In one embodiment the pool of target-specific forward primers comprises primers complementary to approximately 50 different primer binding sites within the target gene or genome of interest. In one embodiment the pool of target-specific forward primers comprises primers complementary to approximately 60 different primer binding sites within the target gene or genome of interest. In one embodiment the pool of target-specific forward primers comprises primers complementary to approximately 70 different primer binding sites within the target gene or genome of interest. In one embodiment the pool of target-specific forward primers comprises primers complementary to approximately 75 different primer binding sites within the target gene or genome of interest. In one embodiment the pool of target-specific forward primers comprises primers complementary to approximately 80 different primer binding sites within the target gene or genome of interest. In one embodiment the pool of target-specific forward primers comprises primers complementary to approximately 90 different primer binding sites within the target gene or genome of interest. In one embodiment the pool of target-specific forward primers comprises primers complementary to approximately 100 different primer binding sites within the target gene or genome of interest. In one embodiment the pool of target-specific forward primers comprises primers complementary to approximately 200 different primer binding sites within the target gene or genome of interest. In one embodiment the pool of target-specific forward primers comprises primers complementary to approximately 300 different primer binding sites within the target gene or genome of interest. In one embodiment the pool of target-specific forward primers comprises primers complementary to approximately 400 different primer binding sites within the target gene or genome of interest. In one embodiment the pool of target-specific forward primers comprises primers complementary to approximately 500 different primer binding sites within the target gene or genome of interest. In one embodiment the pool of target-specific forward primers comprises primers complementary to approximately 600 different primer binding sites within the target gene or genome of interest. In one embodiment the pool of target-specific forward primers comprises primers complementary to approximately 700 different primer binding sites within the target gene or genome of interest. In one embodiment the pool of target-specific forward primers comprises primers complementary to approximately 800 different primer binding sites within the target gene or genome of interest. In one embodiment the pool of target-specific forward primers comprises primers complementary to approximately 900 different primer binding sites within the target gene or genome of interest. In one embodiment the pool of target-specific forward primers comprises primers complementary to approximately 1,000 different primer binding sites within the target gene or genome of interest. In another embodiment the pool of target-specific forward primers comprises primers complementary to approximately 2,000 different primer binding sites within the target gene or genome of interest. In another embodiment the pool of target-specific forward primers comprises primers complementary to approximately 3,000 different primer binding sites within the target gene or genome of interest. In another embodiment the pool of target-specific forward primers comprises primers complementary to approximately 4,000 different primer binding sites within the target gene or genome of interest. In another embodiment the pool of target-specific forward primers comprises primers complementary to approximately 5,000 different primer binding sites within the target gene or genome of interest. In some embodiments the pool of target-specific forward primers comprises primers complementary to from about 50 to about 100 different primer binding sites within the target gene or genome of interest. In some embodiments the pool of target-specific forward primers comprises primers complementary to from about 60 to about 90 different primer binding sites within the target gene or genome of interest. In some embodiments the pool of target-specific forward primers comprises primers complementary to from about 70 to about 80 different primer binding sites within the target gene or genome of interest.

In some such embodiments such compositions comprise a pool of target-specific forward primers complementary to primer binding sites within the target gene or genome of interest that are spaced approximately 25 nucleotides apart. In some such embodiments such compositions comprise a pool of target-specific forward primers complementary to primer binding sites within the target gene or genome of interest that are spaced approximately 50 nucleotides apart. In some such embodiments such compositions comprise a pool of target-specific forward primers complementary to primer binding sites within the target gene or genome of interest that are spaced approximately 75 nucleotides apart. In some such embodiments such compositions comprise a pool of target-specific forward primers complementary to primer binding sites within the target gene or genome of interest that are spaced approximately 100 nucleotides apart. In some such embodiments such compositions comprise a pool of target-specific forward primers complementary to primer binding sites within the target gene or genome of interest that are spaced approximately 125 nucleotides apart. In some such embodiments such compositions comprise a pool of target-specific forward primers complementary to primer binding sites within the target gene or genome of interest that are spaced approximately 150 nucleotides apart. In some such embodiments such compositions comprise a pool of target-specific forward primers complementary to primer binding sites within the target gene or genome of interest that are spaced approximately 175 nucleotides apart. In some such embodiments such compositions comprise a pool of target-specific forward primers complementary to primer binding sites within the target gene or genome of interest that are spaced approximately 200 nucleotides apart.

In some such embodiments the NGS based adapter in the primers in the pool is an Ilumina adapter.

In some such embodiments the primers in the pool are complementary to primer binding sites located within an HPV gene or genome. In some such embodiments the primers in the pool are complementary to primer binding sites located within and HPV circulating tumor DNA (ctDNA). In some such embodiments the HPV ctDNA is from an HPV-associated squamous cell carcinoma of the head and neck, oropharynx, cervix, vulva, vagina, anal canal or penis. In some such embodiments the HPV ctDNA is from HPV type 16, 18, 31, 33, 35, 39, 45, 51, 52, 56, 58 or 59. In some such embodiments the primers in the pool are complementary to primer binding sites the HPV genome that are conserved between HPV strains and sub-strains.

In some such embodiments the pool of target-specific forward comprises one or more of SEQ ID NO. 1 to SEQ ID NO. 77. In some such embodiments the pool of target-specific forward primers comprises at least 10 primers from among SEQ ID NO. 1 through SEQ ID NO. 77. In some such embodiments the pool of target-specific forward primers comprises at least 20 primers from among SEQ ID NO. 1 through SEQ ID NO. 77. In some such embodiments the pool of target-specific forward primers comprises at least 30 primers from among SEQ ID NO. 1 through SEQ ID NO. 77. In some such embodiments the pool of target-specific forward primers comprises at least 40 primers from among SEQ ID NO. 1 through SEQ ID NO. 77. In some such embodiments the pool of target-specific forward primers comprises at least 50 primers from among SEQ ID NO. 1 through SEQ ID NO. 77. In some such embodiments the pool of target-specific forward primers comprises at least 60 primers from among SEQ ID NO. 1 through SEQ ID NO. 77. In some such embodiments the pool of target-specific forward primers comprises at least 70 primers from among SEQ ID NO. 1 through SEQ ID NO. 77. In some such embodiments the pool of target-specific forward primers comprises each of SEQ ID NO. 1 through SEQ ID NO. 77. In some embodiments the pool of target-specific forward primers comprises primers that bind to a target sequence in the HPV genome that: (i) is within the E6 and/or E7 region of the HPV genome, and/or (ii) is 100% conserved between European and non-European HPV isolates.

Each of the compositions described herein can, in some embodiments, comprise one or more additional components compatible with the storage and/or use of the pool of primers, such as suitable salts, buffers, preservatives, nucleotides, enzymes, and the like.

### Applications

The methods and compositions provided herein have a variety of applications. For example, such methods and compositions can be employed to screen for, quantify and/or sequence tumor DNA (such DNA from an HPV-associated tumor) in the circulation of a subject (e.g. a patient suspected of having cancer, such as an HPV-associated cancer). Similarly, in some embodiments such methods and compositions can be employed to assess tumor burden (for example of an HPV-positive tumor) in a subject—as the quantity of the amplified library products should correlate to tumor burden. In some of such methods, controls and/or standard curves are used to quantify or give an estimate of the tumor burden—e.g. in terms of tumor volume or number or tumor cells, etc.

Similarly, the methods and compositions of the present invention can be employed to monitor the progression or recurrence of a cancer (such as an HPV-positive cancer) in a subject, or to monitor the response to therapy of cancer (such as an HPV-positive cancer) in a subject. Such methods involve determining changes in the quantity of the specific amplified library products over time. Typically, such methods entail performing the methods described herein using two or more plasma samples obtained from the subject at different time points. For example, in some embodiments the methods of the present invention are performed using a first plasma sample obtained from a subject at a first time point and a second plasma sample obtained from the subject at a second time point. Using such methods, an increase or decrease in the quantity of the specific amplified library products between the first sample/time point and the second sample/time point can be detected and quantified. For example, an increase in the quantity of the amplified library products between the first sample/time point the second sample/time point may indicate an increase tumor burden, for example as a result of tumor progression, or as a result of tumor recurrence following a previous treatment. Similarly, a decrease in the quantity of the specific amplified library products between a first sample/time point prior to treatment (or earlier in treatment) and a second sample/time point subsequent to commencement of treatment (or later in treatment, or after treatment) may indicate that the treatment is effective. Conversely, an increase in the quantity of the specific amplified library products between a first sample/time point prior to treatment (or earlier in treatment) and a second sample/time point subsequent to commencement of treatment (or later in treatment, or after treatment) may indicate that the treatment is ineffective. In those methods aimed monitoring the response to therapy, the methods may be performed using both a “test” sample and a “control” sample. For example, the test sample may be obtained from a subject treated with a new/test therapeutic molecule and the control sample may be obtained from an untreated subject, or a subject treated with a placebo, or a subject treated with a comparator therapeutic molecule. Such methods can be used to monitor the response to any desired type of therapy, including, but not limited to, therapy with chemotherapeutic agents, therapy with other therapeutic molecules, therapy using radiation, and surgical therapy.

One of skill in the art will recognize that the various methods and compositions of the present invention described throughout this patent disclosure can be combined in various different ways, and that such combinations are within the scope of the present invention.

One of skill in the art will also recognize that the methods and compositions of the present invention described herein are applicable more widely than to only detection of circulating tumor DNA and to only detection in plasma samples, but can also be applied to, and used in conjunction with, detection of various other forms of DNA (i.e. other than ctDNA) and various other tissue samples (i.e. other than plasma), including, but not limited to, blood, urine, cerebrospinal fluid, saliva, and cervical tissue samples. Thus, in each instance in the present specification, and the accompanying claims, in which an embodiment of the invention is described as involving a plasma sample, the present invention also encompasses the analogous embodiment in which another tissue sample (such as blood, urine, cerebrospinal fluid, saliva, or a cervical sample) is used in place of the plasma sample. Similarly, in each instance in the present specification, and the accompanying claims, in which an embodiment of the invention is described as involving ctDNA, the present invention also encompasses the analogous embodiment in which another type of source of DNA (i.e. other than ctDNA) is used or detected.

The invention is further described by the following non-limiting “Examples,” as well as the Figures referred to therein and the descriptions of such Figures provided above.

### EXAMPLES

### Example 1

### “SPECTRE-Seq” for Improved Detection and Monitoring of HPV Associated Cancers

The present example demonstrates the development of a modified single primer extension (SPEX) technique termed “SPECTRE-seq” which is useful for the detection and sequencing of ctDNA and other forms of cfDNA. In the present non-limiting example, this technique is used to capture the entire HPV genome from a ctDNA sample for high throughput sequencing. However, the SPECTRE-seq method can also be applied to detection of other target sequences of interest in ctDNA, cfDNA or other sources of fragmented DNA.

Rationale: cfDNA is fragmented (106-200 bp) and conventional PCR approaches capture only a fraction of cfDNA that contains opposing primer sequences. Without a multiplex strategy to assay for HPV, PCR based methods cannot estimate the absolute number of number of HPV copies, nor capture multiple loci from the same sample.

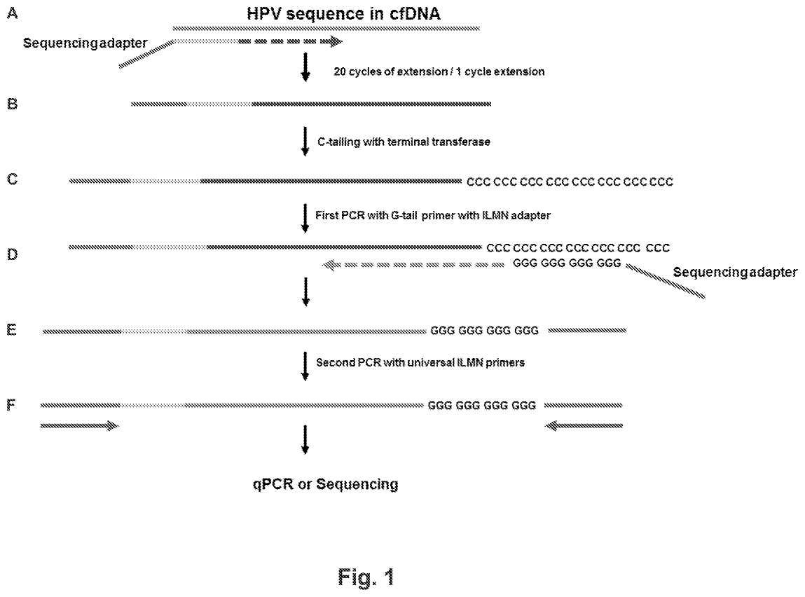

Experimental Design: SPEX was previously developed to generate strand specific accurate sequence information from ancient DNA. (1) SPEX can overcome the limitations of amplifying fragmented DNA by using only one sequence-specific primer per target sequence. We modified prior SPEX methods to improve their utility, sensitivity and specificity for detection of cfDNA and ctDNA. A schematic representation of one of our improved SPECTRE-seq methods is provided in FIG. 1. The primers used for the initial step (FIG. 1A) can amplify highly fragmented DNA without the need for a predefined amplicon size based on a primer pair. The versatility of this technique allows us to efficiently target any region of the genome. The SPECTRE primer extension continues until the end of the fragmented cfDNA (FIG. 1B). These first-generation copies of HPV DNA are then subjected to poly-C tailing using terminal transferase (FIG. 1C). A nested G-rich primer concatenated with another sequencing adapter is used in the reverse direction to make a complete DNA molecule that can be sequenced on any NGS technology based on the adapter sequences and downstream modifications for library preparation (FIG. 1D-F). The final output is sequence information for a combination of HPV sequences—or any other genes of interest based on amplicon design. The resulting sequencing libraries capture cfDNA of varying lengths from plasma without the need for additional hybridization probes or adapter ligation steps.

These SPECTRE-seq methods include several modifications over and above prior SPEX techniques. One modification is the addition of next generation sequencing (NGS)-based adapters on the 5′ ends of both the forward (target-specific) and reverse (not target-specific) primers. This enables the generation of an NGS library without downstream ligation or amplification steps. Another modification is the use of a set of multiple different target-specific forward primers (i.e. a “primer set” or “primer pool”) that tiles across the entirety of the target sequence to be detected. In this particular non-limiting example the primer set comprised primers that tile across the entirety of the 8 kb HPV genome.

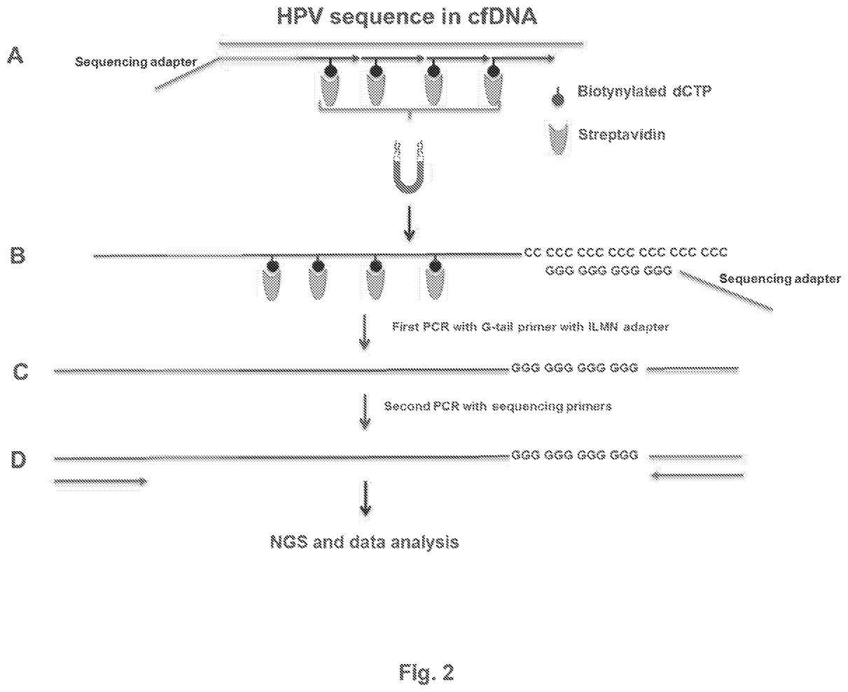

In an additional improvement we also developed a version of this technique in which the SPEX extension reaction is supplemented with biotinylated nucleotides (biotin-dCTP) (as shown schematically in FIG. 2). Using this additional modification the extension reaction streptavidin (SA) beads can be used to exclusively select for and purify the extension/amplification products.

Preliminary results: We optimized the extension, C-tailing, and biotin-SA purification portion of the protocol using one primer and a single stranded DNA template with an HPV sequence.

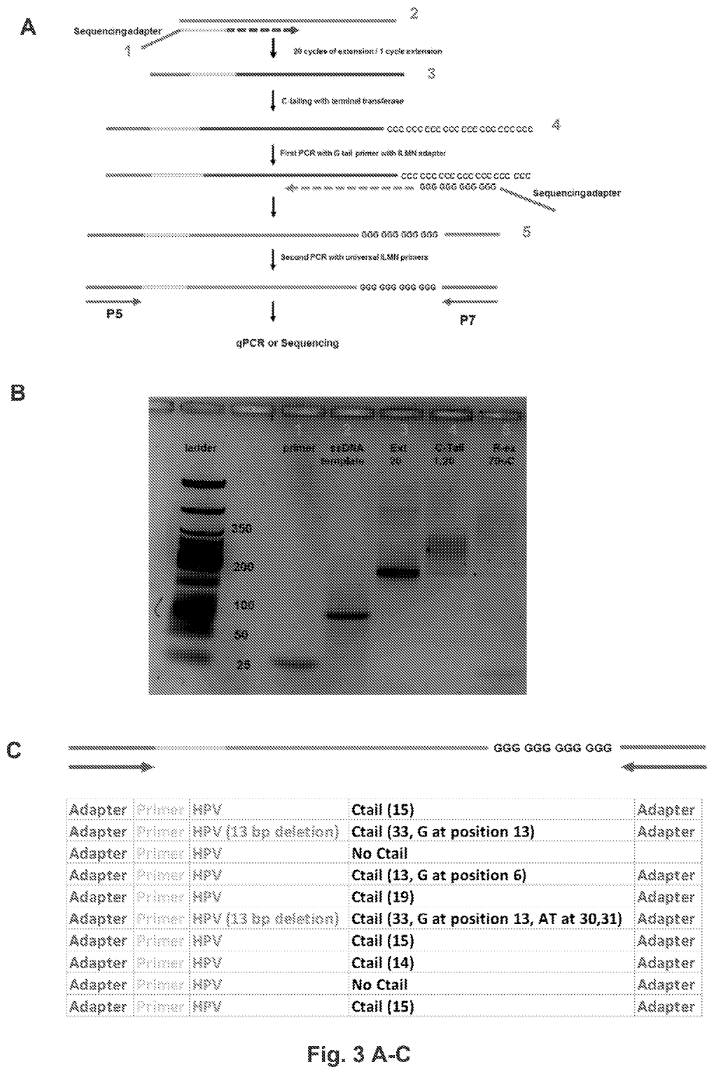

FIG. 3 demonstrates the steps of the SPECTRE-seq assay and the successfully generated product with specifically amplified HPV sequence. The numbers on top of each lane in FIG. 3B correspond to the product specified by the number in FIG. 3A. The final product obtained from this SPECTRE-seq protocol was cloned into the TOPO cloning vector and transformed into One Shot TOP10 chemically competent E. coli strains followed by sanger sequencing of the resultant colonies. FIG. 3C shows sequencing data from 10 colonies with SPECTRE-seq products. We were able to obtain HPV DNA sequence, C-tail and sequencing adapters from all the colonies upon sanger sequencing providing “proof of concept” for the SPECTRE-seq technique.

The key advantages of the SPECTRE-seq technique include: (1) the ability to multiplex across a large sequence (such as the HPV genome), (2) in the case of HPV, the ability to utilize relevant high risk HPV strains, such as 16, 18, 33, and 35, simultaneously, and (3) the potential to incorporate additional genomic regions of interest for detection of cancer mutations.

### Example 2

### “SPECTRE-Seq” Primers for Detection & Sequencing of the HPV16 Genome

Table 1, below, provides an exemplary pool of SPECTRE primers designed to tile across the HPV16 genome—for use in the SPECTRE-seq methods described herein.

TABLE 1

| Amplicon | Forward primer |
|---|---|
| (SEQ ID NO.) | (nucleotide sequence) |
| 1 | tataaaactaagggcgtaacc |
| 2 | aatgtttcaggacccaca |
| 3 | cagttactgcgacgtgag |
| 4 | gacattattgttatagtttgtatgga |
| 5 | aagcaaagacatctggaca |
| 6 | ttgcagatcatcaagaaca |
| 7 | tgcaaccagagacaactg |
| 8 | ggacagagcccattacaa |
| 9 | gggcacactaggaattgt |
| 10 | gggatgtaatggatggttt |
| 11 | atttaacacaggcagaaaca |
| 12 | gcagtacaggttctaaaacga |
| 13 | agagctgcaaaaaggaga |
| 14 | gactgaaacaccatgtagtca |
| 15 | acactatatgccaaacacca |
| 16 | ggggtgagtttttcagaat |
| 17 | gctgacagtataaaaacactattaca |
| 18 | caattgaaaaattgctgtcta |
| 19 | gcagcagcattatattggtat |
| 20 | catttgaattatcacagatgg |
| 21 | aatgcaagtgcctttctaa |
| 22 | aaatgagtatgagtcaatggata |
| 23 | aagatttttgcaaggcata |
| 24 | aatttctgcaagggtctg |
| 25 | tagcagatgccaaaatagg |
| 26 | caactaaaatgccctcca |
| 27 | tggtggtgtttacatttcc |
| 28 | tggtccagattaagtttgc |
| 29 | agtacagacctacgtgaccata |
| 30 | gccaacactggctgtatc |
| 31 | aagtggacattacaagacgtt |
| 32 | tgcagtttgatggagaca |
| 33 | atgttcatgaaggaatacga |
| 34 | ctgtgtttagcagcaacg |
| 35 | accgaagaaacacagacg |
| 36 | acaccactaagttgttgcac |
| 37 | agtaacactacacccatagtacatt |
| 38 | tggacaggacataatgtaaaa |
| 39 | aaataccaaaaactattacagtgtc |
| 40 | taatacgtccgctgcttt |
| 41 | cctctgcgtttaggtgtt |
| 42 | gacacaaacgttctgcaa |
| 43 | cctaaggttgaaggcaaa |
| 44 | gggttaggaattggaaca |
| 45 | agaccccctttaacagtagat |
| 46 | ccccagatgtatcaggatt |
| 47 | cactttcactgacccatct |
| 48 | ttatgaagaaattcctatggatac |
| 49 | atagtcgcacaacacaacag |
| 50 | gcatatgaaggtatagatgtgg |
| 51 | taggccagcattaacctc |
| 52 | ttgatcctgcagaagaaat |
| 53 | tgatatttatgcagatgactttatt |
| 54 | tcaggttatattcctgcaaa |
| 55 | atagttccagggtctccac |
| 56 | ctctttggctgcctagtg |
| 57 | tatgttgcacgcacaaac |
| 58 | atcaggattacaatacagggta |
| 59 | cctgtgtaggtgttgaggt |
| 60 | ggatgacacagaaaatgct |
| 61 | ccacctataggggaacact |
| 62 | cacagttattcaggatggtg |
| 63 | tccagattatattaaaatggtgtc |
| 64 | ggtgaaaatgtaccagacg |
| 65 | ttttcctacacctagtggttc |
| 66 | tgttggggtaaccaactatt |
| 67 | acatggggaggaatatga |
| 68 | cactattttggaggactgg |
| 69 | acacctccagcacctaaa |
| 70 | ttcctttaggacgcaaat |
| 71 | tacaactgctaaacgcaaa |
| 72 | gtgcttgtaaatattaagttgtatgt |
| 73 | attgtgtcatgcaacataaata |
| 74 | aaacttgtacgtttcctgct |
| 75 | gcactatgtgcaactactgaa |
| 76 | gcacatatttttggcttgt |
| 77 | atttgtaaaactgcacatgg |

### REFERENCES

1. Brotherton, P. et al. Novel high-resolution characterization of ancient DNA reveals C &gt; U-type base modification events as the sole cause of post-mortem miscoding lesions. Nucleic Acids Research 35, 5717-5728, doi:10.1093/nar/gkm588 (2007).
2. Brotherton, P., Sanchez, J. J., Cooper, A. & Endicott, P. Preferential access to genetic information from endogenous hominin ancient DNA and accurate quantitative SNP-typing via SPEX. Nucleic Acids Research 38, e7-e7, doi:10.1093/nar/gkp897 (2010).

1. A method of generating a library of fragments of a target gene or genome of interest from a sample of fragmented DNA, wherein the library is suitable for use in detecting, quantifying and/or sequencing the target gene or genome of interest, the method comprising:
(a) Contacting a sample of fragmented DNA with a pool of target-specific forward primers complementary to multiple different primer binding sites located within, and spanning the length of, a target gene or genome of interest, wherein each target-specific forward primer comprises: (i) a sequence that is complementary to a primer binding site within the target gene or genome of interest and (ii) a first next generation sequencing (NGS) based adapter located 5′ to the sequence that is complementary to the primer binding site,
(b) Performing a single primer extension reaction to generate first-generation copies of the target gene of genome of interest,
(c) Adding a common sequence to the 3′ end of the first-generation copies of the target gene or genome of interest, thereby generating 3′ tagged first generation copies of the fragmented target gene or genome of interest,
(d) Performing a first PCR reaction using: a common reverse primer comprising: (i) a sequence that is complementary to the common sequence and (ii) a second next generation sequencing (NGS) based adapter located 5′ to the sequence that is complementary to the common sequence, and
(e) Performing a second PCR reaction using: (i) a forward primer complementary to the NGS-based adapter present in the target-specific forward primer, and (ii) a reverse primer complementary to the NGS-based adapter present the common reverse primer,
thereby generating a library of fragments of a target gene or genome of interest from a sample of fragmented DNA, wherein the library is suitable for use in detecting, quantifying and/or sequencing the target gene or genome of interest.

2. The method of claim 1, wherein the single primer extension reaction of step (b) is performed in the presence of biotinylated nucleotides such that the first-generation copies of the target gene of genome of interest generated by the single primer extension reaction are biotinylated, (d), a biotin-based selection step is performed to select for only biotinylated nucleic acid molecules.

3. The method of claim 1 or claim 2, further comprising performing next generation sequencing of the library of fragments of the target gene or genome of interest.

4. The method of claim 1 or claim 2, further comprising performing quantitative PCR of the library of fragments of the target gene or genome of interest.

5. The method of any of the preceding claims, wherein the sample of fragmented DNA is circulating cell free DNA (cfDNA).

6. The method of any of the preceding claims, wherein the sample of fragmented DNA is circulating tumor DNA (ctDNA).

7. The method of any of the preceding claims, wherein the pool of target-specific forward primers comprises primers complementary to approximately 1,000, or 2,000, or 3,000, or 4,000, or 5,000 different primer binding sites within the target gene or genome of interest.

8. The method of any of the preceding claims, wherein the different primer binding sites within the target gene or genome of interest to which the pool of target-specific forward primers is complementary are spaced approximately 25-200 nucleotides apart.

9. The method of any of the preceding claims, wherein in step (b) 1-99 cycles of the single primer extension reaction are performed.

10. The method of any of the preceding claims, wherein in step (d) 1-99 cycles of the first PCR reaction are performed.

11. The method of any of the preceding claims, wherein in step (e) 1-99 cycles of the second PCR reaction are performed.

12. The method of any of the preceding claims, wherein the NGS based adapter is an Ilumina adapter.

13. The method of any of the preceding claims, wherein the common sequence is a polyC, polyG, polyA or polyT sequence.

14. The method of claim 2, wherein the biotinylated nucleotides are biotin-dCTP, biotin-dGTP, biotin-dATP, biotin dTTTP, or a combination thereof.

15. The method of any of the preceding claims, wherein the sample of fragmented DNA is HPV circulating tumor DNA (ctDNA) and wherein the target gene or genome of interest is an HPV gene or genome.

16. The method of claim 15, wherein the HPV ctDNA is from an HPV-associated squamous cell carcinoma of the head and neck, oropharynx, cervix, vulva, vagina, anal canal or penis.

17. The method of any of claims 15-16, wherein the HPV ctDNA is from HPV type 16, 18, 31, 33, 35, 39, 45, 51, 52, 56, 58 or 59.

18. The method of any of claims 15-17, wherein the pool of target-specific forward primers comprises primers that bind to a target sequence in the HPV genome that: (i) is within the E6 and/or E7 region of the HPV genome, and/or (ii) is 100% conserved between European and non-European HPV isolates.

19. The method of any of claims 15-18, wherein the pool of HPV-specific forward primers comprises one or more of SEQ ID NO. 1 through SEQ ID NO. 77.

20. The method of any of claims 15-18, wherein the pool of HPV-specific forward primers comprises SEQ ID NO. 1 through SEQ ID NO. 77.

21. A composition comprising a pool of target-specific forward primers suitable for use in a single primer extension reaction, wherein the pool comprises primers complementary to multiple different primer binding sites located within, and spanning the length of, a target gene or genome of interest, wherein each target-specific forward primer comprises: (i) a sequence that is complementary to a primer binding site within the target gene or genome of interest and (ii) a first next generation sequencing (NGS) based adapter located 5′ to the sequence that is complementary to the primer binding site.

22. The composition of claim 21, wherein the target gene or genome of interest is present in circulating cell free DNA (cfDNA).

23. The composition of claim 21, wherein the target gene or genome of interest is in circulating tumor DNA (ctDNA).

24. The composition of claim 21, wherein the pool of target-specific forward primers comprises primers complementary to approximately 75 different primer binding sites within the target gene or genome of interest.

25. The composition of claim 21, wherein the wherein the different primer binding sites within the target gene or genome of interest to which the pool of target-specific forward primers is complementary are spaced approximately 100 nucleotides apart.

26. The composition of claim 21, wherein the wherein the NGS based adapter is an Ilumina adapter.

27. The composition of any of claims 21-26, wherein the target gene or genome of interest is in HPV circulating tumor DNA (ctDNA).

28. The composition of claim 27, wherein the HPV ctDNA is from an HPV-associated squamous cell carcinoma of the head and neck, oropharynx, cervix, vulva, vagina, anal canal or penis.

29. The composition of claim 27 or claim 28, wherein the HPV ctDNA is from HPV type 16, 18, 31, 33, 35, 39, 45, 51, 52, 56, 58 or 59.

30. The composition of any of claims 27-29, wherein the pool of target-specific forward primers comprises primers that bind to target sequences in the HPV genome that are conserved between HPV strains and sub-strains.

31. The composition of any of claims 27-30, wherein the pool of HPV-specific forward primers comprises one or more of SEQ ID NO. 1 to SEQ ID NO. 77.

32. The composition of any of claims 27-30, wherein the pool of HPV-specific forward primers comprises SEQ ID NO. 1 to SEQ ID NO. 77.

33. The method of claim 1 wherein the target gene or genome of interest is an HPV 16 gene or genome add wherein the pool of HPV-specific forward primers comprises one or more of SEQ ID NO. 1 through SEQ ID NO. 77.

34. The method of claim 1 wherein the target gene or genome of interest is an HPV 16 gene or genome add wherein the pool of HPV-specific forward primers comprises SEQ ID NO. 1 through SEQ ID NO. 77.


SEQUENCE LISTING (77 sequences)

SEQ ID NO: 1 - DNA, 21 bp, Artificial Sequence
  Description of Artificial Sequence Synthetic primer
tataaaactaagggcgtaacc

SEQ ID NO: 2 - DNA, 18 bp, Artificial Sequence
  Description of Artificial Sequence Synthetic primer
aatgtttcaggacccaca

SEQ ID NO: 3 - DNA, 18 bp, Artificial Sequence
  Description of Artificial Sequence Synthetic primer
cagttactgcgacgtgag

SEQ ID NO: 4 - DNA, 26 bp, Artificial Sequence
  Description of Artificial Sequence Synthetic primer
gacattattgttatagtttgtatgga

SEQ ID NO: 5 - DNA, 19 bp, Artificial Sequence
  Description of Artificial Sequence Synthetic primer
aagcaaagacatctggaca

SEQ ID NO: 6 - DNA, 19 bp, Artificial Sequence
  Description of Artificial Sequence Synthetic primer
ttgcagatcatcaagaaca

SEQ ID NO: 7 - DNA, 18 bp, Artificial Sequence
  Description of Artificial Sequence Synthetic primer
tgcaaccagagacaactg

SEQ ID NO: 8 - DNA, 18 bp, Artificial Sequence
  Description of Artificial Sequence Synthetic primer
ggacagagcccattacaa

SEQ ID NO: 9 - DNA, 18 bp, Artificial Sequence
  Description of Artificial Sequence Synthetic primer
gggcacactaggaattgt

SEQ ID NO: 10 - DNA, 19 bp, Artificial Sequence
  Description of Artificial Sequence Synthetic primer
gggatgtaatggatggttt

SEQ ID NO: 11 - DNA, 20 bp, Artificial Sequence
  Description of Artificial Sequence Synthetic primer
atttaacacaggcagaaaca

SEQ ID NO: 12 - DNA, 21 bp, Artificial Sequence
  Description of Artificial Sequence Synthetic primer
gcagtacaggttctaaaacga

SEQ ID NO: 13 - DNA, 18 bp, Artificial Sequence
  Description of Artificial Sequence Synthetic primer
agagctgcaaaaaggaga

SEQ ID NO: 14 - DNA, 21 bp, Artificial Sequence
  Description of Artificial Sequence Synthetic primer
gactgaaacaccatgtagtca

SEQ ID NO: 15 - DNA, 20 bp, Artificial Sequence
  Description of Artificial Sequence Synthetic primer
acactatatgccaaacacca

SEQ ID NO: 16 - DNA, 19 bp, Artificial Sequence
  Description of Artificial Sequence Synthetic primer
ggggtgagtttttcagaat

SEQ ID NO: 17 - DNA, 26 bp, Artificial Sequence
  Description of Artificial Sequence Synthetic primer
gctgacagtataaaaacactattaca

SEQ ID NO: 18 - DNA, 21 bp, Artificial Sequence
  Description of Artificial Sequence Synthetic primer
caattgaaaaattgctgtcta

SEQ ID NO: 19 - DNA, 21 bp, Artificial Sequence
  Description of Artificial Sequence Synthetic primer
gcagcagcattatattggtat

SEQ ID NO: 20 - DNA, 21 bp, Artificial Sequence
  Description of Artificial Sequence Synthetic primer
catttgaattatcacagatgg

SEQ ID NO: 21 - DNA, 19 bp, Artificial Sequence
  Description of Artificial Sequence Synthetic primer
aatgcaagtgcctttctaa

SEQ ID NO: 22 - DNA, 23 bp, Artificial Sequence
  Description of Artificial Sequence Synthetic primer
aaatgagtatgagtcaatggata

SEQ ID NO: 23 - DNA, 19 bp, Artificial Sequence
  Description of Artificial Sequence Synthetic primer
aagatttttgcaaggcata

SEQ ID NO: 24 - DNA, 18 bp, Artificial Sequence
  Description of Artificial Sequence Synthetic primer
aatttctgcaagggtctg

SEQ ID NO: 25 - DNA, 19 bp, Artificial Sequence
  Description of Artificial Sequence Synthetic primer
tagcagatgccaaaatagg

SEQ ID NO: 26 - DNA, 18 bp, Artificial Sequence
  Description of Artificial Sequence Synthetic primer
caactaaaatgccctcca

SEQ ID NO: 27 - DNA, 19 bp, Artificial Sequence
  Description of Artificial Sequence Synthetic primer
tggtggtgtttacatttcc

SEQ ID NO: 28 - DNA, 19 bp, Artificial Sequence
  Description of Artificial Sequence Synthetic primer
tggtccagattaagtttgc

SEQ ID NO: 29 - DNA, 22 bp, Artificial Sequence
  Description of Artificial Sequence Synthetic primer
agtacagacctacgtgaccata

SEQ ID NO: 30 - DNA, 18 bp, Artificial Sequence
  Description of Artificial Sequence Synthetic primer
gccaacactggctgtatc

SEQ ID NO: 31 - DNA, 21 bp, Artificial Sequence
  Description of Artificial Sequence Synthetic primer
aagtggacattacaagacgtt

SEQ ID NO: 32 - DNA, 18 bp, Artificial Sequence
  Description of Artificial Sequence Synthetic primer
tgcagtttgatggagaca

SEQ ID NO: 33 - DNA, 20 bp, Artificial Sequence
  Description of Artificial Sequence Synthetic primer
atgttcatgaaggaatacga

SEQ ID NO: 34 - DNA, 18 bp, Artificial Sequence
  Description of Artificial Sequence Synthetic primer
ctgtgtttagcagcaacg

SEQ ID NO: 35 - DNA, 18 bp, Artificial Sequence
  Description of Artificial Sequence Synthetic primer
accgaagaaacacagacg

SEQ ID NO: 36 - DNA, 20 bp, Artificial Sequence
  Description of Artificial Sequence Synthetic primer
acaccactaagttgttgcac

SEQ ID NO: 37 - DNA, 25 bp, Artificial Sequence
  Description of Artificial Sequence Synthetic primer
agtaacactacacccatagtacatt

SEQ ID NO: 38 - DNA, 21 bp, Artificial Sequence
  Description of Artificial Sequence Synthetic primer
tggacaggacataatgtaaaa

SEQ ID NO: 39 - DNA, 25 bp, Artificial Sequence
  Description of Artificial Sequence Synthetic primer
aaataccaaaaactattacagtgtc

SEQ ID NO: 40 - DNA, 18 bp, Artificial Sequence
  Description of Artificial Sequence Synthetic primer
taatacgtccgctgcttt

SEQ ID NO: 41 - DNA, 18 bp, Artificial Sequence
  Description of Artificial Sequence Synthetic primer
cctctgcgtttaggtgtt

SEQ ID NO: 42 - DNA, 18 bp, Artificial Sequence
  Description of Artificial Sequence Synthetic primer
gacacaaacgttctgcaa

SEQ ID NO: 43 - DNA, 18 bp, Artificial Sequence
  Description of Artificial Sequence Synthetic primer
cctaaggttgaaggcaaa

SEQ ID NO: 44 - DNA, 18 bp, Artificial Sequence
  Description of Artificial Sequence Synthetic primer
gggttaggaattggaaca

SEQ ID NO: 45 - DNA, 21 bp, Artificial Sequence
  Description of Artificial Sequence Synthetic primer
agaccccctttaacagtagat

SEQ ID NO: 46 - DNA, 19 bp, Artificial Sequence
  Description of Artificial Sequence Synthetic primer
ccccagatgtatcaggatt

SEQ ID NO: 47 - DNA, 19 bp, Artificial Sequence
  Description of Artificial Sequence Synthetic primer
cactttcactgacccatct

SEQ ID NO: 48 - DNA, 24 bp, Artificial Sequence
  Description of Artificial Sequence Synthetic primer
ttatgaagaaattcctatggatac

SEQ ID NO: 49 - DNA, 20 bp, Artificial Sequence
  Description of Artificial Sequence Synthetic primer
atagtcgcacaacacaacag

SEQ ID NO: 50 - DNA, 22 bp, Artificial Sequence
  Description of Artificial Sequence Synthetic primer
gcatatgaaggtatagatgtgg

SEQ ID NO: 51 - DNA, 18 bp, Artificial Sequence
  Description of Artificial Sequence Synthetic primer
taggccagcattaacctc

SEQ ID NO: 52 - DNA, 19 bp, Artificial Sequence
  Description of Artificial Sequence Synthetic primer
ttgatcctgcagaagaaat

SEQ ID NO: 53 - DNA, 25 bp, Artificial Sequence
  Description of Artificial Sequence Synthetic primer
tgatatttatgcagatgactttatt

SEQ ID NO: 54 - DNA, 20 bp, Artificial Sequence
  Description of Artificial Sequence Synthetic primer
tcaggttatattcctgcaaa

SEQ ID NO: 55 - DNA, 19 bp, Artificial Sequence
  Description of Artificial Sequence Synthetic primer
atagttccagggtctccac

SEQ ID NO: 56 - DNA, 18 bp, Artificial Sequence
  Description of Artificial Sequence Synthetic primer
ctctttggctgcctagtg

SEQ ID NO: 57 - DNA, 18 bp, Artificial Sequence
  Description of Artificial Sequence Synthetic primer
tatgttgcacgcacaaac

SEQ ID NO: 58 - DNA, 22 bp, Artificial Sequence
  Description of Artificial Sequence Synthetic primer
atcaggattacaatacagggta

SEQ ID NO: 59 - DNA, 19 bp, Artificial Sequence
  Description of Artificial Sequence Synthetic primer
cctgtgtaggtgttgaggt

SEQ ID NO: 60 - DNA, 19 bp, Artificial Sequence
  Description of Artificial Sequence Synthetic primer
ggatgacacagaaaatgct

SEQ ID NO: 61 - DNA, 19 bp, Artificial Sequence
  Description of Artificial Sequence Synthetic primer
ccacctataggggaacact

SEQ ID NO: 62 - DNA, 20 bp, Artificial Sequence
  Description of Artificial Sequence Synthetic primer
cacagttattcaggatggtg

SEQ ID NO: 63 - DNA, 24 bp, Artificial Sequence
  Description of Artificial Sequence Synthetic primer
tccagattatattaaaatggtgtc

SEQ ID NO: 64 - DNA, 19 bp, Artificial Sequence
  Description of Artificial Sequence Synthetic primer
ggtgaaaatgtaccagacg

SEQ ID NO: 65 - DNA, 21 bp, Artificial Sequence
  Description of Artificial Sequence Synthetic primer
ttttcctacacctagtggttc

SEQ ID NO: 66 - DNA, 20 bp, Artificial Sequence
  Description of Artificial Sequence Synthetic primer
tgttggggtaaccaactatt

SEQ ID NO: 67 - DNA, 18 bp, Artificial Sequence
  Description of Artificial Sequence Synthetic primer
acatggggaggaatatga

SEQ ID NO: 68 - DNA, 19 bp, Artificial Sequence
  Description of Artificial Sequence Synthetic primer
cactattttggaggactgg

SEQ ID NO: 69 - DNA, 18 bp, Artificial Sequence
  Description of Artificial Sequence Synthetic primer
acacctccagcacctaaa

SEQ ID NO: 70 - DNA, 18 bp, Artificial Sequence
  Description of Artificial Sequence Synthetic primer
ttcctttaggacgcaaat

SEQ ID NO: 71 - DNA, 19 bp, Artificial Sequence
  Description of Artificial Sequence Synthetic primer
tacaactgctaaacgcaaa

SEQ ID NO: 72 - DNA, 26 bp, Artificial Sequence
  Description of Artificial Sequence Synthetic primer
gtgcttgtaaatattaagttgtatgt

SEQ ID NO: 73 - DNA, 22 bp, Artificial Sequence
  Description of Artificial Sequence Synthetic primer
attgtgtcatgcaacataaata

SEQ ID NO: 74 - DNA, 20 bp, Artificial Sequence
  Description of Artificial Sequence Synthetic primer
aaacttgtacgtttcctgct

SEQ ID NO: 75 - DNA, 21 bp, Artificial Sequence
  Description of Artificial Sequence Synthetic primer
gcactatgtgcaactactgaa

SEQ ID NO: 76 - DNA, 19 bp, Artificial Sequence
  Description of Artificial Sequence Synthetic primer
gcacatatttttggcttgt

SEQ ID NO: 77 - DNA, 20 bp, Artificial Sequence
  Description of Artificial Sequence Synthetic primer
atttgtaaaactgcacatgg